(19) United States
(12) Patent Application Publication
(10) Pub. No.: US 2024/0189468 A1
(43) Pub. Date: Jun. 13, 2024

(54) WIRELESS POWER TRANSFER DEVICE WITH ULTRAVIOLET LIGHT SOURCE

(71) Applicants: robert j. nathanson, san francisco, CA (US); SOPHIA J. BOUTILIER, mill valley, CA (US); HARPER H. BOUTILIER, mill valley, CA (US)

(72) Inventors: robert j. nathanson, san francisco, CA (US); SOPHIA J. BOUTILIER, mill valley, CA (US); HARPER H. BOUTILIER, mill valley, CA (US)

(21) Appl. No.: 18/205,502

(22) Filed: Jun. 3, 2023

Related U.S. Application Data
(60) Provisional application No. 63/432,059, filed on Dec. 13, 2022.
(60) Provisional application No. 63/430,943, filed on Dec. 7, 2022.

(51) Int. Cl. A61L 2/10; H02J 7/00; H02J 7/34; H02J 50/10; H02J 50/90

(52) U.S. Cl. CPC A61L 2/10; H02J 7/0044; H02J 7/342; H02J 50/10; H02J 50/90; A61L 2202/16

(57) ABSTRACT

A method of a wireless power device with an ultraviolet (UV) light source, comprising: providing the wireless power device with the UV light source, wherein wireless power device with the UV light source comprises: a wireless power device configured to wirelessly power another device's battery system, a UV light source configured to emit a UV light that sanitizes a surface; activating the UV light source; exposing the UV light source to the surface. The method includes coupling the wireless power device with the UV light source with the other device's battery system by placing the wireless power device with the UV light source on the other device.

### CLAIM OF PRIORITY

This application claims priority to U.S. Patent Application No. 63/432,059, filed on 13 Dec. 2022, and titled WIRELESS POWER TRANSFER DEVICE WITH ULTRAVIOLET LIGHT SOURCE. This provisional application is hereby incorporated by reference in its entirety.

### BACKGROUND

Viruses can live on surfaces for up to five (5) days, so devices or surfaces which come into regular contact with or are shared between people can provide a higher risk. Additionally, micro-organisms such as bacteria, molds, yeasts and protozoa can be destroyed or removed by physical, biological and chemical methods. These can have harmful or harsh side effects on the users and can be harmful to the environment creating waste and harmful emissions in its manufacture, transportation and use. They can also damage the surfaces being cleaned.

UV-C works using a photolytic effect whereby the radiation inactivates the micro-organism so that it can no longer multiply. For DNA, it does this by causing adjacent thymine bases to form a chemical bond thus creating a dimmer and if sufficient of these are created, DNA cannot replicate. UV-C disinfection uses light to break down the chemical compounds in a micro-organism like bacteria, spores and viruses so they cannot multiply. Over the years, many hundreds of bacteria have been tested, and all of them respond to UV-C disinfection3—including various coronaviruses. UV-C light sources reduced SARS-COV-2 virus infectivity on a surface to below detectable levels in as few as nine (9) seconds. In this way, UV-C light should play a key role in a COVID-19 protection strategy.

### SUMMARY OF THE INVENTION

A method of a wireless power device with an ultraviolet (UV) light source, comprising: providing the wireless power device with the UV light source, wherein wireless power device with the UV light source comprises: a wireless power device configured to wirelessly power another device's battery system, a UV light source configured to emit a UV light that sanitizes a surface; activating the UV light source; exposing the UV light source to the surface. The method includes coupling the wireless power device with the UV light source with the other device's battery system by placing the wireless power device with the UV light source on the other device.

### BRIEF DESCRIPTION OF THE DRAWINGS

The present application can be best understood by reference to the following description taken in conjunction with the accompanying figures, in which like parts may be referred to by like numerals.

FIGS. 1 and 2 illustrate an example embodiment of a wireless power device with UV light source, according to some embodiments.

FIG. 3 shows WPT-UV attached to a mobile device while in a wireless charging mode, according to some embodiments.

FIG. 4 illustrates an example process for sanitizing a surface with a WPT-UV, according to some embodiments.

FIG. 5 illustrates an example process for charging a mobile device battery system with a WPT-UV, according to some embodiments.

FIG. 6 depicts an exemplary computing system that can be configured to perform any one of the processes provided herein.

FIG. 7 illustrates an example exploded view of a wireless power device with UV light source, according to some embodiments.

FIG. 8 shows another example of a WPT-UV attached to a mobile device while in a wireless charging mode, according to some embodiments.

The Figures described above are a representative set and are not an exhaustive with respect to embodying the invention.

### DESCRIPTION

Disclosed are a system, method, and article of manufacture of a wireless power transfer device with ultraviolet light source. The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments.

Reference throughout this specification to “one embodiment,” “an embodiment,” “one example,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### Definitions

The following terminology is used in example embodiments:

Mobile device can be a computer small enough to hold and operate in the hand. Mobile devices typically have a flat LCD or OLED screen, a touchscreen interface, and digital or physical buttons. Many such devices can connect to the Internet and connect with other devices such as car entertainment systems or headsets via Wi-Fi, Bluetooth, cellular networks or near field communication (NFC). Integrated cameras, the ability to place and receive voice and video telephone calls, video games, and Global Positioning System (GPS) capabilities are common. Power is typically provided by a lithium-ion battery. Mobile devices may run mobile operating systems that allow third-party applications to be installed and run. As used herein, a mobile device can be a smart phone, tablet computer, computerized medical device, other handheld computer(s), etc.

Ultraviolet (UV) is a form of electromagnetic radiation with wavelength from 10 nm (e.g. with a corresponding frequency around 30 PHz) to 400 nm (e.g. 750 THz). It is noted that one example embodiment uses an approximately 200 nm UV-C light.

Ultraviolet C (UV-C) light can be between 100-280 nm. UV-C light can be used for, inter alia: short-wave UV, germicidal UV, and/or ionizing radiation at shorter wavelengths.

Wireless power transfer (WPT) can include, inter alia: wireless power transmission, wireless energy transmission (WET), or electromagnetic power transfer, and the like. WPT can be the transmission of electrical energy without wires as a physical link. In a wireless power transmission system, a transmitter device, driven by electric power from a power source, generates a time-varying electromagnetic field, which transmits power across space to a receiver device, which extracts power from the field and supplies it to an electrical load. In some examples herein, WPT systems can be near field (e.g. a non-radiative technique, etc.) of power transfer. In a near-field system the power can be transferred over a short distance by magnetic fields using inductive coupling between coils of wire, or by electric fields using capacitive coupling between metal electrodes.

### Example Systems and Methods

In one example, a portable light sanitizer/wireless charging device(s) is provided (e.g. a wireless power device with UV light source as discussed infra, a Kr—Cl excimer lamp(s), etc.). The portable light sanitizer/wireless charging device can have an additional feature for magnetically connecting to and charging wirelessly and/or magnetically hand-held mobile communication devices and tablets such as cellular/smart phones. The portable light sanitizer/wireless charging device(s) can have one or more radiation emitters including, inter alia, one or more ultraviolet (UV-C) light emitters. These can be configured for personal, hand-held-ready disinfecting, sanitizing and germicidal purposes. In one example, the portable light sanitizer/wireless charging device can include a UV-C LED with a wavelength of approximately 265 nm inactivate more than 99.9% of viruses and bacteria.

Accordingly, in one embodiment, the portable light sanitizer/wireless charging device can combine the various light treatment modalities discussed herein with the benefits of ubiquitous mobile communication devices in the form a portable device that magnetically attaches to mobile communication devices to empower users to protect against germs in a quick, easy, and portable manner when on the go.

The portable light sanitizer/wireless charging device(s) can be used to conveniently sterilize pathogenic microorganisms from personal contact items such as counter tops, public transportation surfaces, telephones, key pads, computer keyboards and screens, and computer pointer devices, automated teller machine (ATM) touch screens, desk tops, automotive surfaces, gasoline pumps, airplane surfaces, desks, theater surfaces, restaurant surfaces and utensils, nail care and other personal care utensils, cutting boards, keys, eye glasses, other personal items, toilet seats and flush handles, sinks faucet handles and knobs, children's toys, and/or gym equipment. The portable light sanitizer/wireless charging device(s) can be used to sanitize minor cuts, wounds, etc.

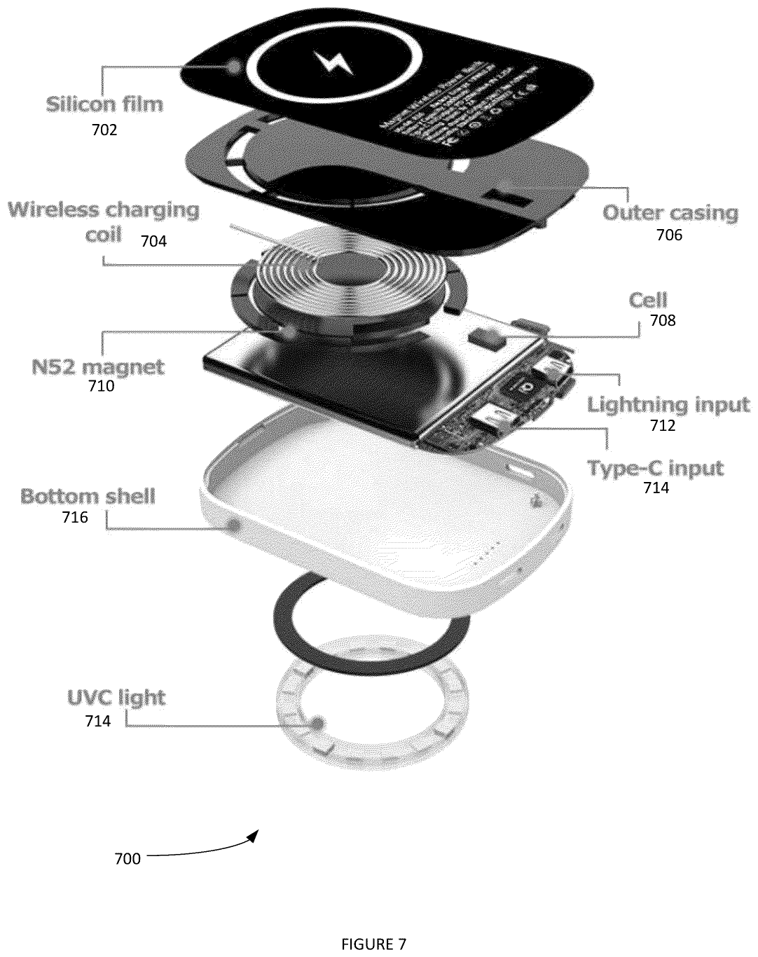

In one example embodiment, the portable light sanitizer/wireless charging device (e.g. see FIG. 7 infra) can be magnetically attached to or removed from a mobile electronic device. The invention can also be used on its own without a mobile device. At least one light source can be installed and configured to emit electromagnetic radiation in the range of ultraviolet light from the back of the invention.

In this way, the light source can be configured to emit electromagnetic radiation safe for humans having a wavelength. This wavelength can be in the range of approximately 220-1000 nanometers for a timed duration. In some examples, this wavelength can be far-UVC light (e.g. 207-222 nm). Far-UVC light (207-222 nm) can efficiently kills pathogens potentially without harm to exposed human tissues. In one example, this can be at a 222 nm far-UVC light wavelength. In some examples, the light source's maximum output can be set at 222 nm.

The light source(s) can be held at various distance from a surface to be disinfected. In one example, this can be at 10 cm (e.g. 6 mW/cm²), the UV light can kill >99.9% of viruses and bacteria. Particles close to the light (e.g. 2 cm away), may be subject to a much higher dosage (e.g. 150 mW/cm²). At this distance, it would only take 0.5 s to kill over 99.9% of viruses and bacteria. Thus at 5 cm, it may be used for 4-5 seconds to kill over 99.9% viruses and bacteria.

A power source within the portable device is configured to power at least one light source and can also serve to recharge a mobile device. The invention also has the capability to be plugged into a mobile electronic device for charging via a power connector.

In one example embodiment, the portable light sanitizer/wireless charging device can include instruments configured for specific surface types. In some examples, the portable light sanitizer/wireless charging device can have a form that is designed for specific surface shapes. For example, a dental instrument can be included that itself includes a light sanitizer source. This example version can be shaped to fit into a user's mouth and shine light on the interior surfaces.

Additional, embodiments are now discussed with reference to the figures.

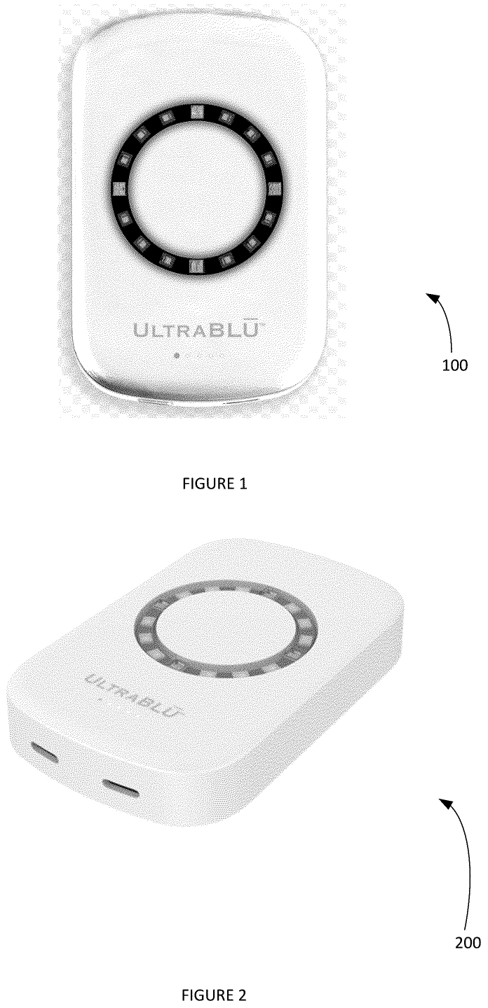

FIGS. 1 and 2 illustrate an example embodiment of a wireless power device with UV light source 100 (WPT-UV 100 as used herein), according to some embodiments. WPT-UV 100 can be used for, inter alia: air, surface and object disinfection using UV-C light. WPT-UV 100 can inactivate SARS-COV-2 on surfaces by more than 99% to below detectable levels. The disinfection effect is directly related to the UV-C dose and leaves no chemical residue or smells, unlike liquid cleaning processes.

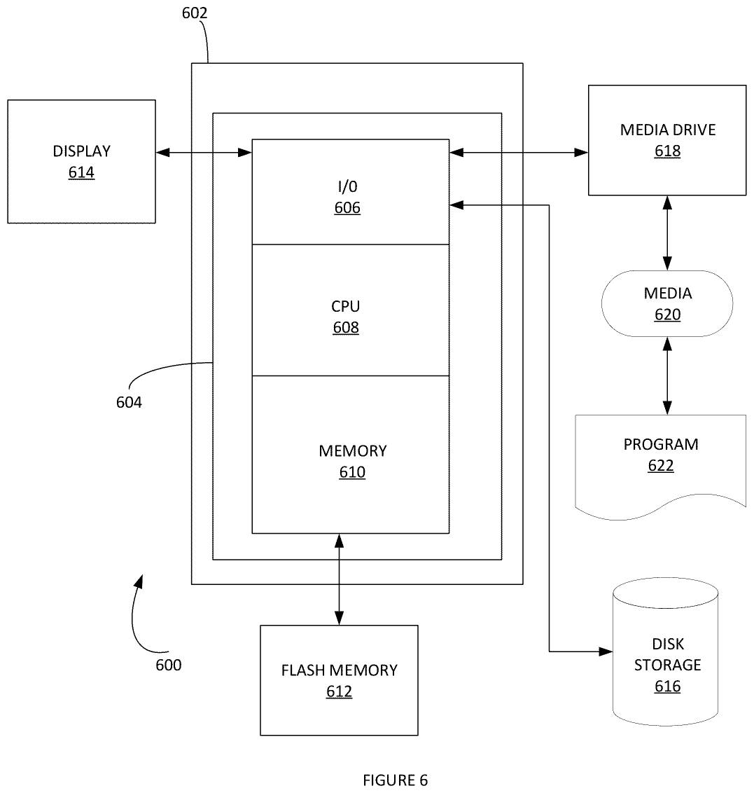

WPT-UV 100 can include a UV-C light source(s). WPT-UV 100 can include a wireless charging system. WPT-UV 100 can include a power source. WPT-UV 100 can include an interface for turning on the UV-C light source(s) and/or other radiation emitters. WPT-UV 100 can include a computing system (e.g. see FIG. 6 infra) for managing the operation of the wireless charging system, the UV-C light source(s), maintain usage logs, notification of power remaining, etc. WPT-UV 100 can include a coupling system for attaching to a mobile device. The coupling system can be a magnetic coupling system, mechanical coupling system, adhesive coupling system, and/or any combination thereof.

FIG. 1 shows the UV-C light source(s) of WPT-UV 100 in an off state. FIG. 2 shows the UV-C light source(s) of WPT-UV 100 in an on state.

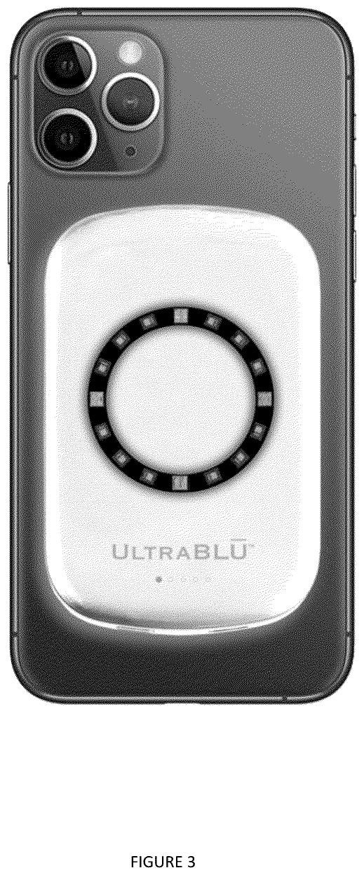

FIG. 3 shows screenshot of a WPT-UV 100 magnetically attached to a mobile device while in a wireless charging mode, according to some embodiments. Here, WPT-UV 100 can be driven by electric power from its own battery power source. When in this mode, WPT-UV 100 can generate a time-varying electromagnetic field and transmits power across space to the receiving mobile device, which in turn extracts power from the field and supplies it to its own battery system. WPT-UV 100 can implement a near field (e.g. a non-radiative technique, etc.) of power transfer.

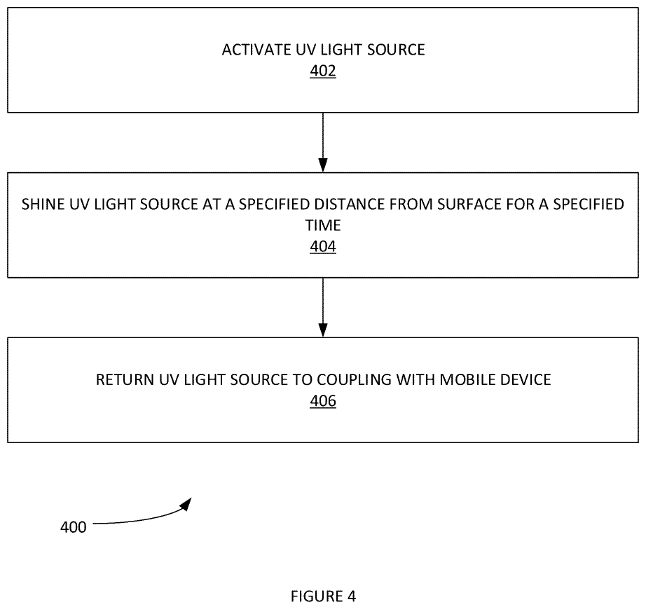

FIG. 4 illustrates an example process 400 for sanitizing a surface with a WPT-UV, according to some embodiments. In step 402, process 400 can activate UV light source. In step 404, process 400 can shine UV light source at a specified distance from surface for a specified time. In step 406, process 400 can return UV light source to coupling with mobile device.

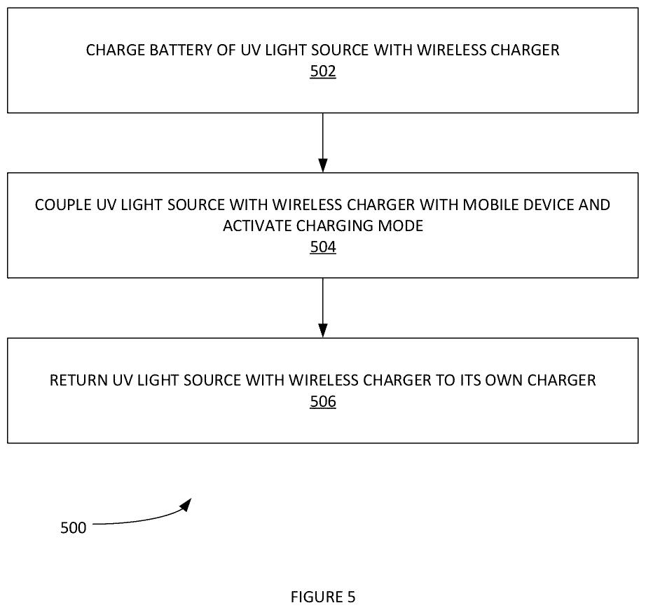

FIG. 5 illustrates an example process 500 for charging a mobile device battery system with a WPT-UV, according to some embodiments. In step 502, process 400 can charge battery of UV light source with wireless charger. In step 504, process 400 can couple UV light source with wireless charger with mobile device and activate charging mode. In step 506, process 400 can return UV light source with wireless charger to its own charger.

### Additional Computing Systems

FIG. 6 depicts an exemplary computing system 600 that can be configured to perform any one of the processes provided herein. In this context, computing system 600 may include, for example, a processor, memory, storage, and I/O devices (e.g., monitor, keyboard, disk drive, Internet connection, etc.). However, computing system 600 may include circuitry or other specialized hardware for carrying out some or all aspects of the processes. In some operational settings, computing system 600 may be configured as a system that includes one or more units, each of which is configured to carry out some aspects of the processes either in software, hardware, or some combination thereof.

FIG. 6 depicts computing system 600 with a number of components that may be used to perform any of the processes described herein. The main system 602 includes a motherboard 604 having an I/O section 606, one or more central processing units (CPU) 608 and/or graphical processing unit (GPU), and a memory section 610, which may have a flash memory card 612 related to it. The I/O section 606 can be connected to a display 614, a keyboard and/or another user input (not shown), a disk storage unit 616, and a media drive unit 618. The media drive unit 618 can read/write a computer-readable medium 620, which can contain programs 622 and/or databases. Computing system 600 can include a web browser. Moreover, it is noted that computing system 600 can be configured to include additional systems in order to fulfill various functionalities. Computing system 600 can communicate with other computing devices based on various computer communication protocols such a Wi-Fi, Bluetooth® (and/or other standards for exchanging data over short distances includes those using short-wavelength radio transmissions), USB, Ethernet, cellular, an ultrasonic local area communication protocol, etc.

### Additional Views

FIG. 7 illustrates an example exploded view 700 of a wireless power device with UV light source, according to some embodiments. Exploded 700 view illustrates a schematic showing the relationship and order of assembly of various elements of an example wireless power device with UV light source.

Exploded 700 view includes silicon film 702. Silicon film 702 is coupled with outer casing 706. Silicon film 702 can be an elastomer (e.g. a rubber-like material) composed of a silicone polymer. It is noted that in other example embodiments, other polymers can be utilized in lieu of a silicone polymer.

Outer casing 706 encases wireless charging coil 704. Wireless charging coil 704 can implement a wireless power transfer with a power course of a coupled mobile device. Wireless charging coil 704 can create a magnetic field which induces a current in another coil (e.g. in another mobile device, cell phone, etc.) charging its battery.

Wireless charging coil 704 is coupled with N52 magnet 710. N52 magnet 710 can be an N52 grade neodymium magnet. N52 magnet contains an energy product and/or (BH)Max of 52MGOe (Mega-Gauss Oersteds). It is noted that various magnet grades can be utilized in other example embodiments (e.g. grades N35, N42, N52, or N55, etc.).

Cell 708 provides power inputs via cell 708. Cell 708 includes lighting input 712 and Type-C input 714. Cell 708 is encased by bottom shell 716. Cell 708 includes a central processing unit (CPU). The CPU can be programmed to implement various operations and steps discussed herein. The CPU can be a microprocessor fabricated on a metal-oxide-semiconductor (MOS) integrated circuit (IC) chip. Cell 708 can include sensors that enable the CPU to detect a coupled mobile device. In this way, CPU can place wireless power device with UV light source in an activate charging mode. CPU can include programming to manage the wireless charging functionalities. For example, CPU can detect and place the wireless power device with UV light source in an inactivate charging mode when it is detected that the battery of the coupled mobile device is fully charged.

Cell 708 can include a battery that provides a power source for the various wireless power device functions (e.g. including wireless charging operations, LED power supply, CPU operations, haptic operations, etc.). In one example, wireless power device with UV light source uses a lithium-ion battery (LIB), whereas older handsets used nickel-metal hydride (Ni-MH) batteries.

It is noted that outer casing 706 and/or bottom shell 716 can be made of thermoplastic polyurethane (TPU) and/or polycarbonate (PC).

Wireless power device with UV light source can also include other systems. For example, wireless power device with UV light source can include haptics systems, computer clock systems, speakerphone systems, user input/interface systems, Bluetooth® systems, Wi-fi communication systems, etc. For example, haptics or sound alarm when UV light exposure time is complete. Wireless power device with UV light source can also a dual input/output source (e.g. USB-C and/or other computer bus/power connector cables, etc.).

In some examples, wireless power device with UV light source can simultaneously charge multiple units as well. For example, wireless power device with UV light source can wirelessly charge a smart phone attached using a magnetically attached power connector technology while it is wired to another phone using one of the cables (e.g. USB-C and/or other computer bus/power connector cables, etc.).

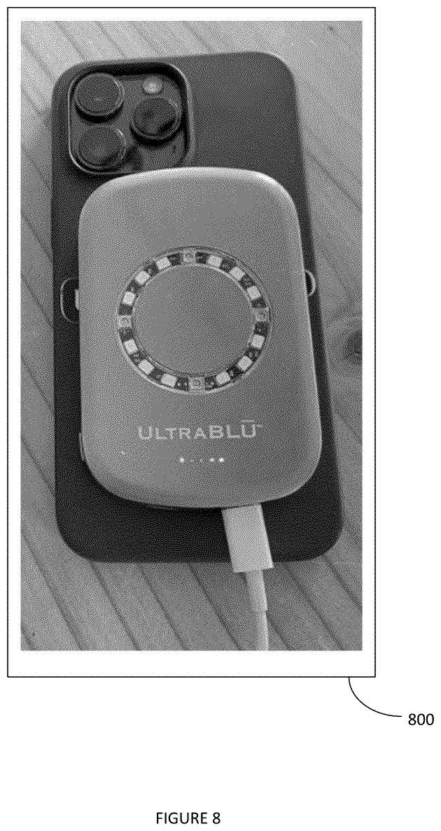

FIG. 8 shows another example of a WPT-UV attached to a mobile device while in a wireless charging mode, according to some embodiments. For example, FIG. 8 shows a wireless power device simultaneously wirelessly charging a mobile device's battery system and a second mobile device's battery system using a computer bus/power connector system.

### CONCLUSION

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g., embodied in a machine-readable medium).

In addition, it can be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

What is claimed by this United States patent:

1. A method of a wireless power device with an ultraviolet (UV) light source, comprising:
providing the wireless power device with the UV light source, wherein wireless power device with the UV light source comprises:
a wireless power device configured to wirelessly power another device's battery system,
a UV light source configured to emit a UV light that sanitizes a surface;
activating the UV light source; and
exposing the UV light source to the surface.

2. The method of claim 1 further comprising:
coupling the wireless power device with the UV light source with the other device's battery system by placing the wireless power device with the UV light source on the other device.

3. The method of claim 2, wherein the wireless power device with the UV light source comprises a wireless charging coil and a battery.

4. The method of claim 3, wherein the UV light source comprises a UV-C light source.

5. The method of claim 4, wherein the UV-C light source emits a UV-C light between one hundred to two-hundred and eighty (100-280) nano meters (nm).

6. The method of claim 5, wherein the UV-C light source emits a UV-C light at two hundred (200) nm.

7. The method of claim 6, wherein the UV-C light source is placed at two to ten (2-10) centimeters from the surface while the UV-C light source emits the 200 nm light.

8. The method of claim 7, wherein the UV-C light source is placed five (5) centimeters from the surface while the UV-C light source emits the 200 nm light for five (5) seconds.

9. The method of claim 8, wherein the wireless power device with the UV light source comprises a haptics system that sends a haptics signal to indicate the surface has been sterilized.

10. The method of claim 9, wherein the haptics system sends the haptics signal to indicate the other device's battery system is fully charged.

11. The method of claim 10 further comprising:
coupling the wireless power device with the UV light source with the other device's battery system by placing the wireless power device with the UV light source on the other device and securing the coupling with an N52 grade neodymium magnet with the wireless power device with the UV light source.

12. The method of claim 10, wherein the N52 grade neodymium magnet is proximate to the wireless charging coil and the battery within the wireless power device with the UV light source.

13. The method of claim 12, wherein the wireless power device with the UV light source comprises a computer bus/power connector system.

14. The method of claim 12, wherein the wireless power device simultaneously wirelessly charges the other device's battery system and a second device's battery system using computer bus/power connector system.
